(19) United States
(12) Patent Application Publication
(10) Pub. No.: US 2025/0382955 A1
(43) Pub. Date: Dec. 18, 2025

(54) DIAPHRAGM PUMP DRIVE

(71) Applicant: FRESENIUS MEDICAL CARE DEUTSCHLAND GMBH, Bad Homburg (DE)

(72) Inventors: Torsten HOCHREIN, Bad Homburg (DE); Martin SCHAMBERGER, Bad Homburg (DE); Frank HEDMANN, Bad Homburg (DE)

(73) Assignee: FRESENIUS MEDICAL CARE DEUTSCHLAND GMBH, Bad Homburg (DE)

(21) Appl. No.: 19/101,905

(22) PCT Filed: Aug. 7, 2023
(86) PCT No.: PCT/EP2023/071811

§ 371 Date: Feb. 7, 2025

(30) Foreign Application Priority Data
Aug. 12, 2022 (DE) 20 2022 104 589.5

(51) Int. Cl. F04B 49/08; A61M 1/14; A61M 1/28; F04B 43/067

(52) U.S. Cl. CPC F04B 49/08; A61M 1/159; A61M 1/281; F04B 43/067

(57) ABSTRACT

The present invention discloses a diaphragm pump drive for driving the pump chamber of a diaphragm pump, said pump drive comprising at least one pressure sensor and comprising a controller, wherein the controller controls the diaphragm pump drive and evaluates measured values from the pressure sensor, wherein the controller is configured to determine a basic compressibility value for the diaphragm pump, wherein the determination of the basic compressibility value is performed on the basis of at least one measurement and preferably a plurality of measurements, during which fluid is located in the pump chamber.

The present invention relates to a diaphragm pump drive.

Diaphragm pumps are often used in the medical technology field, in particular in the field of dialysis technology, to pump medical fluids such as dialysate or blood. In this context, a diaphragm pump conventionally comprises a pump chamber sealed by a diaphragm, whereby fluid can be pushed out of the pump chamber by pressing the diaphragm into the pump chamber, and fluid can be suctioned into the pump chamber by pulling the diaphragm out from the pump chamber. This allows fluid to be pumped through the pump chamber in conjunction with appropriate valves.

The pump chamber is usually arranged in a disposable article such as a pump cassette, which is connected to a diaphragm pump drive. The diaphragm pump drive typically comprises a drive chamber, which is likewise sealed by a diaphragm. The pump chamber and the drive chamber are then coupled to one another such that the diaphragm in the pump chamber follows the movement of the diaphragm in the drive chamber.

In a piston diaphragm pump, the drive chamber is hydraulically connected to a piston-cylinder unit. By moving the piston, hydraulic fluid can be pressed into the drive chamber or suctioned out of it, which results in a corresponding movement of the drive chamber diaphragm. The advantage of such an arrangement is that the pump pressure can be controlled by corresponding control or regulation of the pressure in the hydraulic part. In addition, diaphragm pumps enable straightforward measurement of the pumped fluids, because the volume change in the pump chamber, and thus the fluid displacement during a pump stroke, corresponds (with the opposite sign) to the volume change in the control chamber, with this being precisely determinable via the position of the piston of the piston-cylinder unit.

However, sources of error may occur here. For one thing, air accumulations in the pump chamber may cause the amount of fluid being pumped through the pump chamber to fail to correspond exactly to the volume change in the drive chamber. In addition, due to a certain basic compressibility of the diaphragm pump, the volume change in the drive chamber may deviate from the volume change caused by the movement of the piston-cylinder unit. In particular, air that accumulates in the hydraulic fluid may lead to a certain compressibility in the hydraulic system. Furthermore, hoses and the like that connect the piston-cylinder unit to the drive chamber may have a certain flexibility and may therefore expand under increased pressure. Other drive mechanisms may also have a certain basic compressibility, which influences the values captured for measurement. Additional influences result from the disposable article and the connection thereof.

Known from DE 19919572 A1 is a method by means of which the air content in the fluid being pumped through a pump chamber can be determined. By this method, the pump chamber is first gravimetrically filled, and the resulting initial pressure is measured. The pump chamber shutoff valves are then closed, resulting in a volume of fluid trapped in the pump chamber. With the shutoff valves closed, the piston-cylinder unit is then actuated in order to apply a specified final pressure to the sealed volume of fluid. The volume change of the fluid volume in the pump chamber associated with this pressure change is thus directly related to the air content in the trapped fluid volume. As a result, the air fraction can be determined based on the volume change generated by the pressure difference, which change is determined via the piston movement. In DE 19919572 A1, the influence of the basic compressibility of the diaphragm pump drive, which is referred to therein as system compressibility, is taken into account by means of a fixed, specified constant. However, the basic compressibility may change during operation of the pump, such as due to an accumulation of air in the hydraulic fluid, which is not taken into account in DE 19919572 A1.

A method is therefore known from DE 102011105824 B3 that can determine the basic or system compressibility of a diaphragm pump drive. By this method, the system compressibility of the pump apparatus filled with gas is determined by adjusting an initial and final pressure using a pressure sensor and recording the associated pump positions or pressure sensor values. The spring constant is determined on the basis of the value pairs and equated with the basic or system compressibility.

Publication DE 10 2014 013 152 A1 discloses a method for determining a basic or system compressibility value for a medical diaphragm pump drive, in which method the diaphragm of the diaphragm pump drive is supported on a rigid surface (e.g., the rear wall of the pump chamber or the drive chamber) during determination of the basic or system compressibility value. In this method, the pump chamber is empty or not even connected yet.

The object of the present invention is to provide an improved diaphragm pump drive.

This object is achieved by means of a diaphragm pump drive according to claims 1 and 2. Further preferential embodiments of the invention are the subject matter of the dependent claims.

According to a first aspect, the present invention comprises a diaphragm pump drive for driving the pump chamber of a diaphragm pump, said pump drive comprising at least one pressure sensor and a controller, whereby the controller controls the diaphragm pump drive and evaluates measured values from the pressure sensor, the controller being configured to determine a basic compressibility value for the diaphragm pump. It is provided here that the determination of the basic compressibility value is performed on the basis of at least one measurement and preferably multiple measurements made while fluid is in the pump chamber.

The present invention thus enables determination of the basic compressibility after starting up the diaphragm pump and/or during ongoing operation. The present invention also enables more precise determination of the basic compressibility because the diaphragm pump flexibility resulting from the pump chamber itself is also taken into account. The present invention also takes into account that the basic compressibility has a varying effect on varying chamber volumes. Given that the measurement is performed with fluid in the pump chamber, the procedure according to the invention is better at depicting the basic compressibility across the working cycle of the diaphragm pump.

Furthermore, in cases where the pump chamber is designed as a component of a disposable article and is connected to the diaphragm pump drive, the flexibility of the disposable article and the attachment thereof are also taken into account.

According to a second aspect, the present invention comprises a diaphragm pump drive for driving the pump chamber of a diaphragm pump, said pump drive comprising at least one pressure sensor and a controller, whereby the controller controls the diaphragm pump drive and evaluates measured values from the pressure sensor, the controller being configured to determine a basic compressibility value for the diaphragm pump and/or the air content of the pumped fluid. It is provided here that the determination of the basic compressibility value and/or the air content in the pumped fluid is based on at least two measurements, between which the chamber volume of the pump chamber was changed by suctioning and/or pumping fluid.

This procedure also takes into account that the basic compressibility has a varying effect on varying chamber volumes. Given that at least two measurements are performed, between which the chamber volume of the pump chamber was changed by the suction and/or pumping of fluid, the method according to the invention is better at depicting the basic compressibility across the working cycle of the diaphragm pump.

The diaphragm pump drives according to the first and second aspects are each independently the subject matter of the present invention. However, in a preferred embodiment, the two aspects are combined with one another.

Preferred embodiments of both the first and second aspects are described in greater detail hereinafter.

The basic compressibility value according to the invention can be any desired parameter, by means of which a compressibility property or the flexibility of the diaphragm pump and/or the diaphragm pump drive can be characterized and preferably quantified during pressure changes.

According to one possible embodiment, it is provided that an overall compressibility value for the overall system generated by the diaphragm pump and the fluid in the pump chamber is determined during each of the measurements.

According to one possible embodiment, it is provided that the basic compressibility value for the diaphragm pump and/or the air content is determined by means of a regression analysis of the overall compressibility values, in particular by means of a regression analysis of the overall compressibility values as a function of the chamber volume in the pump chamber, and/or by means of linear regression.

According to one possible embodiment, it is provided that the overall compressibility value is determined as the value which results from the regression analysis for a chamber volume of zero in the pump chamber.

According to one possible embodiment, it is provided that the air content is determined on the basis of a change in the overall compressibility values as a function of the chamber volume in the pump chamber, and in particular based on a slope of a regression line.

According to one possible embodiment, it is provided that the basic compressibility value is determined and referred to in order to determine the air content of the fluid to be pumped in a subsequent measurement, in particular during a later pump cycle, the air content preferably being determined based on only one measurement of the overall compressibility value for the overall system generated by the diaphragm pump and the fluid in the pump chamber, in particular by the measured value corrected via the basic compressibility value.

According to one possible embodiment, it is provided that the basic compressibility value is determined initially, in particular as part of an initial test routine.

According to one possible embodiment, it is provided that the basic compressibility value and/or the air fraction is/are determined during ongoing operation.

According to one possible embodiment, it is provided that the basic compressibility value and/or the air fraction is/are repeatedly determined, and in particular determined during each pump cycle.

According to one possible embodiment, it is provided that the diaphragm pump drive comprises at least one valve drive for driving at least one valve for controlling the flow of liquid into and/or out of the pump chamber, whereby the controller of the diaphragm pump drive controls the at least one valve drive.

According to one possible embodiment, it is provided that controller is configured to control the at least one valve drive in order to perform the at least one measurement, and preferably multiple measurements, according to one of the preceding claims in order to determine the basic compressibility value and/or the air content.

According to one possible embodiment, the diaphragm pump drive comprises a coupling surface to which a pump cassette can be connected, and which comprises the pump chamber and preferably one or more valves.

According to one possible embodiment, it is provided the diaphragm pump drive comprises a drive chamber which is sealed by a diaphragm, whereby the diaphragm is deflected outwards from the drive chamber by positive pressure in the drive chamber, and inwards into the drive chamber by negative pressure in the drive chamber.

According to one possible embodiment, it is provided that the pressure sensor determines the pressure in the drive chamber.

According to one possible embodiment, it is provided that the pressure in the drive chamber is generated via a piston-cylinder unit connected to the drive chamber.

According to one possible embodiment, it is provided that a length sensor is provided which registers the position of the piston, and/or that the transfer of the pressure to the diaphragm is preferably performed hydraulically, whereby the piston cylinder unit and the drive chamber are preferably filled with hydraulic fluid.

According to one possible embodiment, in order to perform a measurement, it is provided that the controller is configured to reach a first and a second pressure level by controlling the diaphragm pump drive when the pump chamber is closed and to detect associated operating parameter values of the diaphragm pump drive, and/or to reach a first and a second operating parameter value by controlling the diaphragm pump drive when the pump chamber is closed and to detect associated pressure levels.

According to one possible embodiment, it is provided that the overall compressibility value is determined on the basis of the operating parameter values and/or pressure levels, whereby the operating parameter values are preferably position values for the diaphragm pump drive.

In one possible embodiment of the present invention, in order to perform a measurement, it is provided that the controller is configured to reach a first and a second pressure level by controlling the diaphragm pump drive when the pump chamber is closed and to detect associated operating parameter values for the diaphragm pump drive.

In order to reach the first and second pressure levels, the diaphragm pump drive is preferably operated until the pressure of the diaphragm pump and/or of the diaphragm pump drive reaches the first pressure level. The first diaphragm pump drive operating parameter value is then determined. The diaphragm pump drive is then operated until the pressure of the diaphragm pump and/or of the diaphragm pump drive reaches the second pressure level, and the second operating parameter value is then determined. The pressure of the diaphragm pump drive and/or the diaphragm pump can thus be measured via the pressure sensor.

Here, the first and second pressure levels can be specified pressure levels. In particular, these can be stored in a controller of the diaphragm pump drive.

In one possible embodiment of the present invention, in order to perform a measurement, the controller is configured to reach a first and a second operating parameter value by controlling the diaphragm pump drive when the pump chamber is closed and to measure the associated pressure levels.

Here, the first and second operating parameter values can be specified values. In particular, these can be stored in a controller of the diaphragm pump drive.

The operating parameters can be determined via a corresponding operating parameter sensor, for example a position and/or motion sensor. The operating parameter values are preferably position values for the diaphragm pump drive.

The overall compressibility value is preferably determined on the basis of the operating parameter values and/or pressure levels.

The present invention further comprises a medical device, in particular a blood treatment machine, in particular a dialysis machine, in particular a peritoneal dialysis machine, having a diaphragm pump drive according to the invention.

In particular, the medical device comprises a pump cassette receptacle and/or an air cushion for pressing the pump cassette onto a coupling surface of the diaphragm pump drive. The controller of the diaphragm pump drive is preferably integrated into the controller of the medical device, in particular the blood treatment machine.

Preferred embodiments of the present invention will now be described in greater detail with reference to drawings and exemplary embodiments:

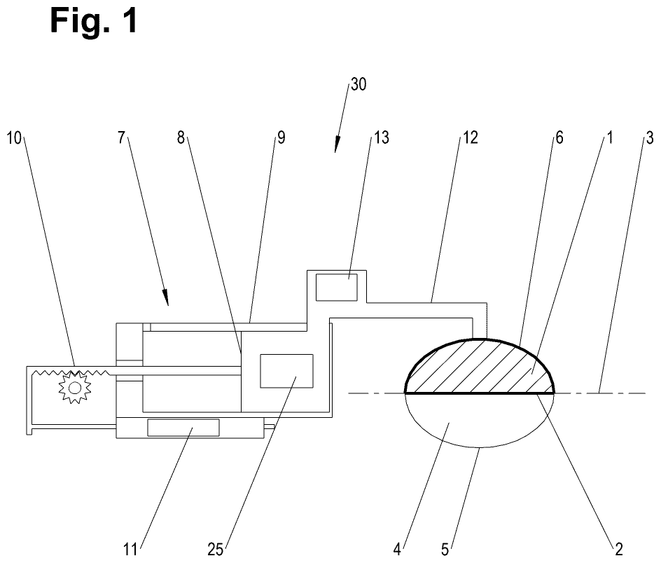

FIG. 1: shows a schematic illustration of a diaphragm pump drive according to the invention comprising a connected pump chamber,

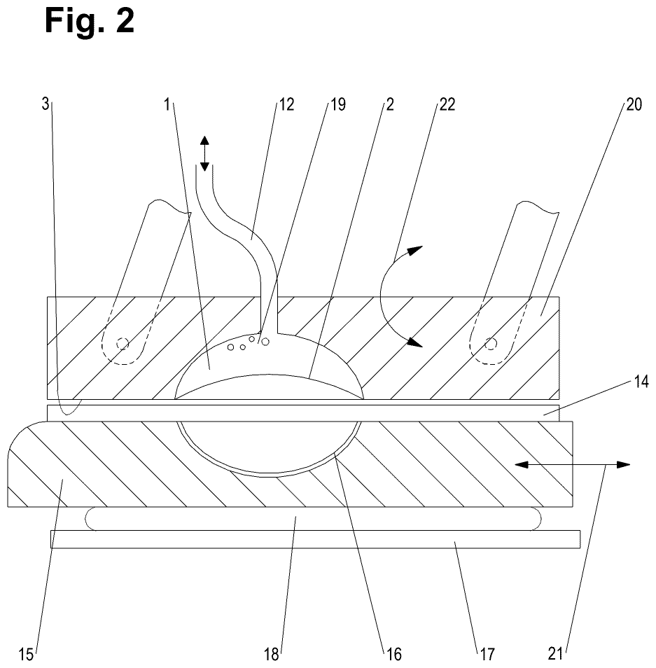

FIG. 2: shows a section through the coupling region of a diaphragm pump drive according to the invention comprising a connected pump cassette,

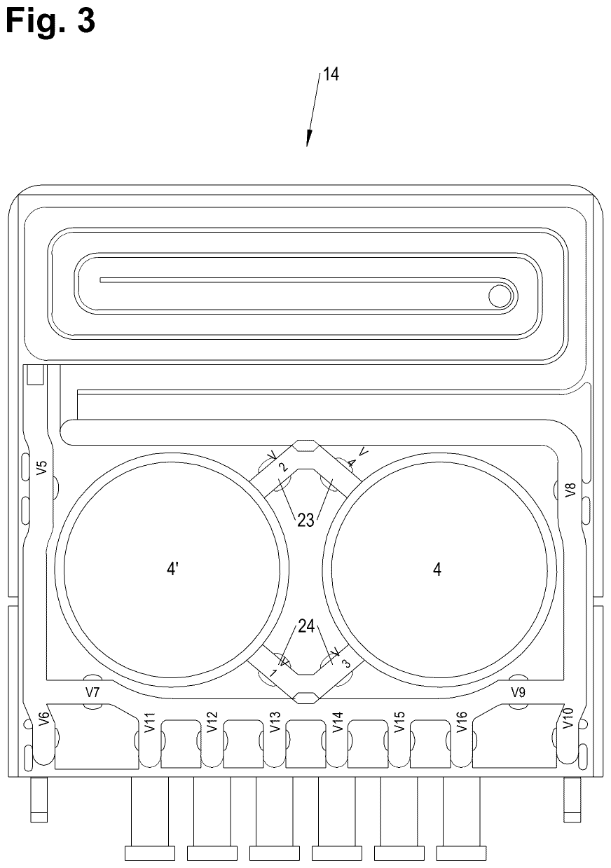

FIG. 3: shows an exemplary embodiment of a pump cassette and the manner in which it can be coupled to a diaphragm pump drive according to the invention, and

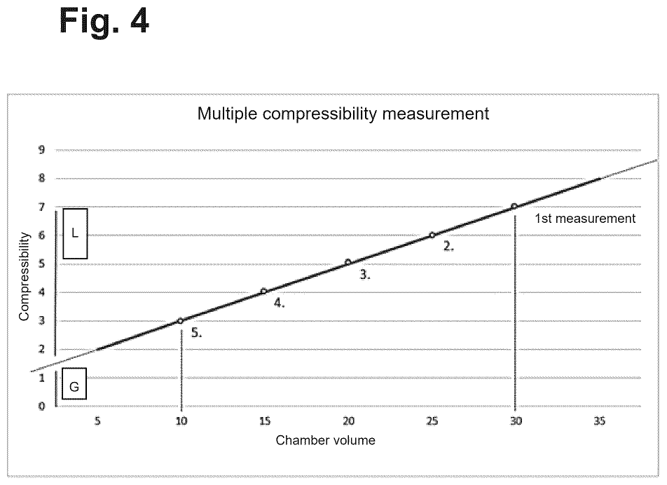

FIG. 4 shows a diagram in which multiple measured values for the overall compressibility are shown as a function of the pump chamber volume in order to explain the present invention.

FIG. 1 shows one exemplary embodiment of a diaphragm pump drive 30 according to the invention for pumping a fluid through the pump chamber 4.

The diaphragm pump drive comprises a drive chamber 1, on which a flexible diaphragm 2 is arranged. The flexible diaphragm 2 is arranged in a coupling surface 3 of the diaphragm pump drive, so that a diaphragm of the pump chamber 4 (not discernible in FIG. 1) can be connected to the diaphragm 2 of the drive chamber such that the former diaphragm follows the movements of the diaphragm 2 of the drive chamber. Via movement of the diaphragm 2 into or out of the drive chamber 1, the volume of the pump chamber 4 can thus be changed. By means of the corresponding switching of valves (not shown in greater detail in FIG. 1) which control the flow into or out of the pump chamber 4, fluid can be pumped by using the pump chamber 4 to move the diaphragm 2.

The pump chamber 4 is conventionally part of a pump cassette (not shown in greater detail in FIG. 1), which preferably represents a disposable article that can be connected to the diaphragm pump drive. The pump chamber is conventionally formed by the corresponding shaping of a hard part of the pump cassette, which is covered by a flexible film that forms the diaphragm of the pump chamber.

However, the present invention would also be usable in the same way in diaphragm pumps in which the drive chamber and the pump chamber are fixedly connected to one another or integrated into a shared pump apparatus.

The embodiment shown in FIG. 1 is a piston diaphragm pump which comprises a piston-cylinder unit 7 that is in hydraulically connected to the drive chamber 6 via the hydraulic line 12. The piston-cylinder unit 7 is driven by a drive 10, which acts on the piston 8 of the piston-cylinder unit 7 and moves the piston within the cylinder 9. The path traveled by the piston 8 within the cylinder 9 is detected and/or measured by a length sensor 11 associated with the piston-cylinder unit 7.

The pressure side 25 of the piston-cylinder unit 7 is fluidly connected to the drive chamber 1 via the fluid line 12, whereby the pressure side 25, the fluid line 12, and the drive chamber 1 are filled with hydraulic fluid. As a result, the actuating movement of the piston 8 is transferred to the diaphragm 2 of the drive chamber 1. Therefore, given a corresponding change of the hydraulic volume of the piston-cylinder unit 7 due to movement of the piston 8, the diaphragm 2 of the drive chamber 1 bulges convexly outwards, or is pulled concavely into the interior of the drive chamber.

The volume change of the drive chamber 1 required for conveying fluid in the pump chamber 4 is accordingly caused by operating the piston-cylinder unit 7. By operating the piston 8, the hydraulic fluid is pressed into or out of the drive chamber 1. This operates the diaphragm 2, the movement of which is transferred to the pump chamber 5 and changes the volume of the latter.

The diaphragm pump drive further comprises a pressure sensor 13, via which the pressure of the hydraulic fluid in the hydraulic system, and thus the pressure in the drive chamber 1, can be measured. The pressure prevailing in the drive chamber 1 corresponds—apart from any counterpressure in the diaphragm 2—to the counterpressure prevailing in the pump chamber 4, so that the pressure in the pump chamber 4 can at the same time be determined via the pressure sensor 13.

The diaphragm pump drive further comprises a controller (not shown), which is connected to the length sensor 11 and the pressure sensor 13 and evaluates the measurement signals. In addition, the controller controls the drive 10 of the diaphragm pump drive, as well as the valves for controlling the flow of fluid into and out of the pump chamber 4. For this purpose, the diaphragm pump drive preferably comprises valve actuators that act on valves, which are preferably also integrated into the pump cassette.

The advantage of such a piston diaphragm pump is that it conveys fluid quantities in a very accurate manner, with the overall conveyed quantity able to be precisely measured, because the pump volume corresponds to the stroke volume of the piston-cylinder unit 7 and is precisely measurable by means of the length sensor 11.

FIG. 2 shows the mechanical structure of one exemplary embodiment of a diaphragm pump drive according to the invention, to which a pump cassette can be connected. The diaphragm pump drive comprises a machine block 20, on which the coupling surface 3 is arranged for connecting the pump cassette 14. The drive chamber 1 provided with the flexible diaphragm 2 is inserted into the coupling surface 3 and is hydraulically connected to the piston-cylinder unit 7 (not shown in greater detail here) via the hydraulic line 12.

The pump cassette 14 is inserted into a pump cassette receptacle 15 for connecting to the coupling surface 3, so that the rear side of the pump cassette supports itself on a receptacle surface of the pump cassette receptacle 15. The receptacle surface comprises a corresponding dome-shaped recess in the region of the pump chamber 4, which is designed as a bulge in the rear side of the pump cassette.

Following insertion of the cassette 14, the cassette receptacle 15 is pressed against the coupling surface 3 via an air cushion 18, which is arranged on the rear side and in turn supports itself on a device wall 17. For this purpose, the air cushion is subjected to a corresponding operating pressure which can be between 1,500 and 2,500 mbar, for example.

In the exemplary embodiment, the pump cassette receptacle 15 is designed as a drawer, which can be retracted and extended in direction 21 in order to insert a cassette. In addition, the machine block 20 can be placed upon the pump cassette 14 in movement direction 22. Following insertion of the drawer 15 and placement of the machine block 20, the air cushion 18 is then pressurized to achieve a secure connection of the pump cassette 14 to the coupling surface 3.

However, as an alternative to the structural design in FIG. 2, the pump cassette receptacle 15 could, for example, also be designed as a door which is opened to insert the pump cassette 14 and closed to mount the pump cassette to the coupling surface. In this case, the air cushion 18 would be integrated into the door.

FIG. 3 shows an exemplary embodiment of a pump cassette 14 comprising two pump chambers 4 and 4′. The pump cassette consists of a hard part in which the fluid-guiding channels and the pump chambers are embedded and which is covered by a flexible film towards the coupling surface. Among other things, the pump cassette comprises valves 23 and 24, via which the flow of fluid into or out of the pump chambers 4 and 4′ can be controlled. The valves are likewise operated via actuators arranged in the machine block 20.

In one possible embodiment, the controller of the diaphragm pump drive features a function by means of which the air content of the fluid being conveyed by the diaphragm pump can be determined. By this function, distortion of the measurement of fluid being conveyed through the pump chamber 4 by air bubbles present in the pump chamber 4 can be prevented.

A measuring phase, which can be interposed with the pumping process at each stroke, can be provided in order to determine the air content or the air quantity. Fluid is initially suctioned into the pump chamber 4 via movement of the diaphragm 2 according to the usual pumping process. The shutoff valves of the pump chamber 4 are then closed so that a sealed fluid volume results and, by activating the drive 10, an initial predetermined pressure level pₐ is reached and the associated position of the piston 8 determined. Subsequently, a second pressure level pₑ is in turn reached by operating the drive 10, and the corresponding position of the piston 8 is likewise determined. If the fluid enclosed in the pump chamber 4 comprises a certain gas content, the latter is compressed by the pressure increase, which corresponds to a resulting change in the volume of the pump chamber 4. This volume difference can be determined by the positions of the piston 9 at the initial and final pressure.

Based on the values thus obtained, the controller calculates the air quantity contained in the pump chamber, i.e., the air volume Vₐₜ contained therein at atmospheric pressure. For this purpose, the controller follows the Boyle-Mariotte law, which is as follows for an isothermal state change (i.e., disregarding any temperature change):

$$p \times V = constant .$$

Various states in the measurement phase can be equated on this basis:

$${V}_{a t} \times {p}_{a t} = {V}_{a} \times {p}_{a} = {V}_{e} \times {p}_{e} .$$

Taking into account that the volume difference V_{diff} is determined by the difference between the initial volume and the final volume, hence V_{diff}=Vₐ−Vₑ, the actual gas volume at atmospheric pressure Vₐₜ can be obtained:

$${V}_{at} = \frac{{V}_{diff}}{( \frac{{p}_{at}}{{p}_{a}} - \frac{{p}_{at}}{{p}_{a} + {p}_{diff}} )}$$

Depending on the specific pumping method being used, this formula must take into account that the pressure measured via the pressure sensor 13 on the hydraulic side of the diaphragm pump may sometimes fail to correspond exactly to the pressure in the pump chamber 4, but may sometimes deviate from this pressure by a certain value due to the inherent tension of the diaphragm 2.

In a first variant of the method, however, the air content can be determined using an undeflected diaphragm 2, so that the influence of the diaphragm is negligible. In a second variant, by contrast, the initial pressure pₐ can be corrected by a differential pressure pₘₑₘ that is attributable to the diaphragm between the hydraulic side and the pump side. This pressure can be stored in the controller, for example. As a result, it is possible to perform the determination of the air content while the diaphragm 2 is being pulled quite far or completely into the drive chamber 1, so that the complete pump volume is utilized. The differential pressure pₘₑₘ between the hydraulic side and the pump side which is attributable to the diaphragm can optionally be determined during the startup phase. Depending on the ratio between the pressures on the hydraulic side and the differential pressure attributable to the diaphragm pₘₑₘ, as well as the required accuracy, the differential pressure pₘₑₘ can optionally also be disregarded.

The volume difference entered into the above formula is determined by the distance traveled by the piston S_{diff} and the area thereof A_{K} during compression from pressure level pₐ to pressure level pₑ.

However, it must be taken into account here that the movement of the piston 8 during the pressure change from pₐ to pₑ is not only attributable to the air volume in the pump chamber 4, because the diaphragm pump drive and the pump chamber itself also exhibit a certain flexibility or basic compressibility under pressure changes. In particular, relevant factors here include air that may accumulate in the hydraulic system, as well as a certain flexibility in the hydraulic line 12. Therefore, given a pressure change from pₐ to pₑ, even if no air at all were contained in the pump chamber 4 and the latter would thus be incompressible, the piston 8 would move a certain distance S₀ due to this basic compressibility alone.

The actual volume Vₐₜ of air contained in the pump chamber 4 is thus obtained by taking into account the basic compressibility value S₀ characterizing the basic compressibility as:

$${V}_{at} = \frac{( {S}_{diff} - {S}_{0} ) \cdot {A}_{k}}{( \frac{{p}_{at}}{{p}_{a}} - \frac{{p}_{at}}{{p}_{a} + {p}_{diff}} )}$$

In one possible embodiment, the controller of the diaphragm pump drive according to the invention features a function by which the basic compressibility value S₀ can be determined. Such a function may in particular be used with a diaphragm pump as described above. However, the function can also be used independently of the specific embodiment of the diaphragm pump described above.

The embodiment of the present invention in this regard is based on the following considerations, which play a particular role in the use of the diaphragm pump drive in the context of medical applications, such as peritoneal dialysis, but are also applicable to diaphragm pump drives in general.

The administration of fluid using the diaphragm pump presents the following requirements for this process.
The amount administered must correspond to an established dosing accuracy.
The fluid is allowed to be mixed with air only to a certain degree.

Conveyance of the fluid can, for example, be implemented using one or two piston diaphragm pumps operating in parallel, such as those as described above. The piston diaphragm pumps are moved using motors; pressures and positions are recorded by suitable sensors.

As described above, this arrangement enables calculation of the compressibility of the fluid contained in a piston stroke by way of two pressure levels.

Determination of the air content of the fluid can be derived from the measured compressibility.

The overall compressibility that is actually measured is composed of various factors: the compressibility of the solution in the pump chamber, and the device properties.

The device properties are derived from the components involved in the pump system. These include:
the air remaining in the hydraulic oil
the air remaining between the pump diaphragm and the film set
the movement of the counter bearing (drawer)
the hydraulic hoses expanding under pressure

These factors form a basic compressibility of the system and negatively affect accurate determination of the air content of the fluid to be pumped. It must also be taken into account that the basic compressibility has a varying strength of effect in accordance with varying chamber volumes.

The present invention is intended to take these effects into account in the calculation.

In principle, the following approaches are conceivable for this purpose, some of which are already known:
a) introducing a device constant for the entire series
b) calculation of a compensation factor for an interval
c) compensating for device properties by means of differential measurement within a chamber stroke
d) combining approaches a), b), and c) to achieve greater system safety

These approaches are described in the following:
a) The basic compressibility is due to device properties that could be design-related. Via the series devices, these device properties could be determined once as a device constant. This device constant could be applicable to all devices.

The advantages of this method, which is known from the prior art, arise from the fact that no initial learning process within the machine is necessary, and no erroneous measurements arise. The disadvantage, however, is that the factor must be selected conservatively. Otherwise, overcompensation would take place when taking all series distributions into account because, as a result of overcompensation, the method would result in a failure to detect air content in the solution.

Therefore, the present invention follows another path, thus comprising the designs described in the following:
b) Determination of a compensation factor by means of an initial learning process:

The following procedure is followed in this case:
1^{st} step: The pump chamber is filled with fluid.
2^{nd} step: The overall compressibility is determined.
3^{rd} step: The chamber volume is reduced by pushing fluid out of the pump chamber.
4^{th} step: Steps 2 and 3 are repeated multiple times.
5^{th} step: The basic compressibility is calculated based on the overall compressibility in steps 2 to 4.

Determination of the basic compressibility is most accurate when said determination is performed at the operating point or using the working fluid. However, a determination at the zero point is also conceivable.

Determination of the overall compressibility is preferably performed as previously described above with respect to the function for determining the air content, i.e., in particular by reaching two pressure levels when the pump chamber volume is closed and determining the associated pump positions.

The air content trapped in the fluid behaves in a deterministic manner. The latter fact enables determination of the basic compressibility based on at least two overall compressibility measurements at different chamber volumes.

As shown in FIG. 4, the basic compressibility of the device can be determined based on a regression line for the measurement results at the intersection of the y-axis and referred to as the dead volume; see region G in FIG. 4. The upper region L is the compressibility of the solution at measurement point 1.

The plausibility of the determination result can be checked by means of performing this measurement repeatedly at different chamber volumes.

On the one hand, the procedure according to the invention enables direct determination (already corrected by the basic compressibility) of the air content from the slope of the resulting regression line, or the region L above the intersection with the y-axis. On the other hand, the basic compressibility can be determined and used in a method for correcting the conventionally determined air fraction, as described above.

Determination of the basic compressibility can be made using the disposable article and the dialysis solution during the treatment and during each pump stroke.

Measurements can be performed repeatedly at different chamber volumes, and the compressibility can be extrapolated.

The advantages of this procedure are the following:
Individual determination of the factor for each device and each disposable article.
Taking series distributions into account places fewer demands on the device components and their tolerances.
Measurement and dosing of the dialysis solution can be performed in a more accurate manner.
Leaks in the disposable article, in the connections, or in the pump hydraulics can be recognized and quantified during each pump stroke. This increases patient safety.

As a disadvantage, it must be taken into account that the dosing inaccuracy and the air content in the dialysis solution depend on the reliability of the initial learning process.
c) Compensation for device characteristics by means of differential measurement within a chamber stroke

### Procedure:

1^{st} step: The pump chamber is filled with fluid.
2^{nd} step: The overall compressibility is determined.
3^{rd} step: The chamber volume is reduced by pushing fluid out of the pump chamber.
4^{th} step: Steps 2 and 3 are repeated multiple times.
5^{th} step: The basic compressibility is calculated based on the overall compressibility in steps 2 to 4.

The outflow can be generated when emptying the chamber (as described in steps 1-5) or when filling the chamber by exchanging fluid volumes.

The advantage of this design lies in the individual determination of the device property for each device, each disposable article, and at each treatment time point, as well as in the fact that taking series distributions into account places lower demands on the device components and the tolerances thereof.

In contrast, one disadvantage of this approach is that repeated compressibility measurement is time-consuming and limits device performance data.

However, the following advantages result regardless of which variant according to the invention is used:
a) Less waste, because the limit values for air volume are now related to the solution. Small chambers are no longer overvalued.
b) Improved dosing accuracy
c) Given that the air detection result no longer depends on the chamber size, improved measurement is demonstrated.

As a result, the basic compressibility value determined according to the invention can be entered in the determination of the air volume of the medical fluid pumped, as described above. This value enables more accurate measurement of the fluids being moved through the diaphragm pump because the air volume in the pumped fluids can be determined more accurately.

Determination of the basic compressibility value can additionally be used to verify the degassing quality of the hydraulic system. For example, once the basic compressibility value exceeds a certain threshold, hydraulic system degassing can be performed or the necessity thereof indicated.

The diaphragm pump drive according to the invention is preferably used in a blood treatment device for pumping medical fluids, in particular for pumping blood or dialysate. Particularly preferably, the diaphragm pump drive according to the invention is used in a dialysis machine, whereby the diaphragm pump is used to pump the dialysate into the abdomen of the patient or to draw dialysate from the abdomen of the patient.

1. A diaphragm pump drive for driving the pump chamber of a diaphragm pump, said pump drive comprising at least one pressure sensor and a controller, wherein the controller controls the diaphragm pump drive and evaluates measured values from the pressure sensor, wherein the controller is configured to determine a basic compressibility value for the diaphragm pump, wherein
the determination of the basic compressibility value is performed on the basis of at least one measurement, during which fluid is located in the pump chamber.

2. The diaphragm pump drive according to claim 1, for driving the pump chamber of a diaphragm pump, said pump drive comprising at least one pressure sensor and a controller, wherein the controller controls the diaphragm pump drive and evaluates measured values from the pressure sensor, wherein the controller is configured to determine a basic compressibility value for the diaphragm pump and/or the air content of the pumped fluid, wherein
the determination of the basic compressibility value and/or the air content of the pumped fluid is performed on the basis of at least two measurements, between which the chamber volume of the pump chamber was changed by suctioning and/or pumping fluid.

3. The diaphragm pump drive according to claim 1, wherein an overall compressibility value for the overall system generated by the diaphragm pump and the fluid in the pump chamber is determined during each of the measurements.

4. The diaphragm pump drive according to claim 3, wherein the basic compressibility value for the diaphragm pump and/or the air content is determined using a regression analysis of the overall compressibility values and/or by means of linear regression.

5. The diaphragm pump drive according to claim 4, wherein the overall compressibility value is determined as the value which results from the regression analysis for a chamber volume of zero in the pump chamber.

6. The diaphragm pump drive according to claim 2, wherein the air content is determined on the basis of a change in the overall compressibility values as a function of the chamber volume in the pump chamber.

7. The diaphragm pump drive according to claim 1, wherein the basic compressibility value is determined and used for determining the air content of the fluid to be pumped during a subsequent measurement, wherein the air content is determined based on only one measurement of the overall compressibility value for the overall system, which is formed by the diaphragm pump and the fluid in the pump chamber.

8. The diaphragm pump drive according to claim 7, wherein the basic compressibility value is determined initially.

9. The diaphragm pump drive according to claim 1, wherein the basic compressibility value and/or the air content is determined during ongoing operation.

10. The diaphragm pump drive according to claim 1, wherein the basic compressibility value and/or the air content is/are repeatably determined.

11. The diaphragm pump drive according to claim 1, wherein the diaphragm pump drive comprises at least one valve drive for driving at least one valve for controlling the flow of fluid into and/or out of the pump chamber, wherein the controller of the diaphragm pump drive controls the at least one valve drive.

12. The diaphragm pump drive according to claim 11, wherein the controller is configured to control the at least one valve drive in order to perform the at least one measurement in order to determine the basic compressibility value and/or the air content.

13. The diaphragm pump drive according to claim 1, having a coupling surface to which a pump cassette is capable of being connected, and which comprises the pump chamber.

14. The diaphragm pump drive according to claim 1, wherein the diaphragm pump drive comprises a drive chamber which is sealed by a diaphragm, wherein the diaphragm is deflected outwards from the drive chamber by positive pressure in the drive chamber, and is deflected inwards into the drive chamber by negative pressure in the drive chamber.

15. The diaphragm pump drive according to claim 14, wherein the pressure sensor determines the pressure in the drive chamber.

16. The diaphragm pump according to claim 14, wherein the pressure in the drive chamber is generated via a piston-cylinder unit which is connected to the drive chamber, wherein a length sensor is provided which detects the position of the piston, and/or wherein the transfer of the pressure to the diaphragm is performed hydraulically, wherein the piston-cylinder unit and the drive chamber are filled with hydraulic fluid.

17. The diaphragm pump drive according to claim 1, wherein, in order to perform a measurement, the controller is configured to reach a first and a second pressure level by controlling the diaphragm pump drive when the pump chamber is closed and to measure associated operating parameter values for the diaphragm pump drive, and/or to reach a first and a second operating parameter value by controlling the diaphragm pump drive when the pump chamber is closed and to detect associated pressure levels, wherein the overall compressibility value is determined on the basis of the operating parameter values and/or the pressure levels, wherein the operating parameter values are position values for the diaphragm pump drive.

18. A peritoneal dialysis machine comprising the diaphragm pump drive according to claim 1, wherein the blood treatment machine comprises a pump cassette receptacle and/or an air cushion for pressing the pump cassette onto a coupling surface of the diaphragm pump drive.

19. A diaphragm pump drive for driving the pump chamber of a diaphragm pump, said pump drive comprising at least one pressure sensor and a controller, wherein the controller controls the diaphragm pump drive and evaluates measured values from the pressure sensor, wherein the controller is configured to determine a basic compressibility value for the diaphragm pump and/or the air content of the pumped fluid, wherein the determination of the basic compressibility value and/or the air content of the pumped fluid is performed on the basis of at least two measurements, between which the chamber volume of the pump chamber was changed by suctioning and/or pumping fluid.

20. The diaphragm pump according to claim 1, wherein the determination of the basic compressibility value is performed on the basis of multiple measurements, during which fluid is located in the pump chamber.
